(19) United States
(12) Patent Application Publication
(10) Pub. No.: US 2021/0128817 A1
(43) Pub. Date: May 6, 2021

(54) NEEDLE UNIT WITH BIOSTATIC CHAMBER

(71) Applicant: Novo Nordisk A/S, Bagsvaerd (DK)

(72) Inventors: Henrik Bengtsson, Taastrup (DK); Vera Pinto Glenting, Copenhagen (DK); Bo Kvolsbjerg, Helsingoer (DK); Brian Jensen, Broenshoej (DK)

(21) Appl. No.: 17/253,073

(22) PCT Filed: Jun. 28, 2019
(86) PCT No.: PCT/EP2019/067396

§ 371 Date: Dec. 16, 2020

(30) Foreign Application Priority Data
Jun. 29, 2018 (EP) 18180827.0

(51) Int. Cl. A61M 5/00; A61M 5/32; A61M 5/34

(52) U.S. Cl. CPC A61M 5/001; A61M 5/326; A61M 2005/3117; A61M 5/3291; A61M 5/34

(57) ABSTRACT

The present invention provides a needle unit (10, 310, 410, 510) having a proximal space (13, 513) adapted to accommodate a portion of a variable volume reservoir, the needle unit (10, 310, 410, 510) comprising: a needle carrier (15, 315, 415, 515), a needle tube (21, 121, 221, 321, 421, 521) being fixed to the needle carrier (15, 315, 415, 515) and comprising a distal needle end (23, 123, 223, 323, 423, 523) for providing fluid communication to an injection site, a needle shield (50, 350, 450, 550) carrying a sealed chamber (38, 138, 238, 338, 438, 538) for accommodating a distal portion of the needle tube (21, 121, 221, 321, 421, 521), the sealed chamber (38, 138, 238, 338, 438, 538) being sealed distally by a penetrable self-sealing septum (39, 139, 239, 339, 439, 539), and a flow channel (24, 124, 224, 324, 424, 524) for establishing fluid communication between the sealed chamber (38, 138, 238, 338, 438, 538) and the proximal space (13, 513), wherein the needle shield (50, 350, 450, 550) and the needle carrier (15, 315, 415, 515) are capable of relative motion between a first relative position in which the sealed chamber (38, 138, 238, 338, 438, 538) houses the distal needle end (23, 123, 223, 323, 423, 523), and a second relative position in which the distal needle end (23, 123, 223, 323, 423, 523) protrudes from the sealed chamber (38, 138, 238, 338, 438, 538) through the penetrable self-sealing septum (39, 139, 239, 339, 439, 539), and wherein the needle tube (21, 121, 221, 321, 421, 521) further comprises a side hole (25, 125, 225, 325, 425, 525), the side hole (25, 125, 225, 325, 425, 525) being in fluid communication with the flow channel (24, 124, 224, 324, 424, 524) and positioned within the sealed chamber (38, 138, 238, 338, 438, 538) when the needle shield (50, 350, 450, 550) and the needle carrier (15, 315, 415, 515) are in the second relative position.

### FIELD OF THE INVENTION

The present invention relates generally to medical devices and more particularly to injection needles for use with drug delivery devices.

### BACKGROUND OF THE INVENTION

Injection systems for self-administering of drugs comprising a pen injection device and an attachable pen needle unit have become increasingly popular due to generally simple and convenient handling patterns. Users of such injection systems are recommended to discard the needle unit after a single injection to minimise the risk of contamination. Hence, in the course of its lifetime the injection device is by default used with multiple needle units.

Needle units are typically wrapped and sealed individually to ensure sterility prior to use. In connection with a dose administration action the user must therefore unwrap the needle unit, mount it on the injection device, perform the injection, dismount it from the injection device, re-wrap or otherwise encapsulate it to prevent needle stick injuries, and finally dispose of it, preferably in a dedicated sharps container.

The readying and subsequent removal of the needle unit is both the most complicated and the most time consuming part of the injection procedure. Especially for young and elderly users the handling of the small items and foils can present a challenge and make the task of injection a bit cumbersome. As a result, some users reuse the needle unit several times. In fact, some users only change the needle unit when the injection device is empty or if the needle for example exhibits clogging or hooking. This reduces the number of times these users have to carry out needle handling activities significantly.

However, it also entails increased risks of both infections and needle stick injuries, the former due to needle contamination and the latter due to the users typically disposing of the original needle unit packaging in connection with the fitting of the needle and therefore do not have this available as receptacle for when they change the needle unit after several times of reuse.

WO 2015/062845 (Novo Nordisk A/S) discloses a needle unit for a pen injection device where a portion of the front needle is housed between injections in a reservoir holding a pre-servative containing liquid. This portion of the front needle is thus cleaned by, and stored in, the preservative containing liquid following each injection action, thereby reducing the risk of microbial contamination. The preservative containing liquid is identical to the drug present in the cartridge and is transferred from the cartridge to the reservoir in connection with a first use of the injection device.

While this needle unit concept allows for safe multiple reuses the various embodiments pre-sented in WO 2015/062845 require either proximal movement of a reservoir wall against the force of a dedicated spring component or proximal movement of the cartridge body relative to the piston in order to fill the reservoir, adding to the complexity and cost of the injection device/needle unit system. Furthermore, since the reservoir is filled only once but the needle unit is intended for multiple reuses, e.g. over a time period of several weeks, the microbe-hostile environment in the reservoir may degrade due to evaporation of components of the preservatives in the preservative containing liquid.

### SUMMARY OF THE INVENTION

It is an object of the invention to eliminate or reduce at least one drawback of the prior art, or to provide a useful alternative to prior art solutions.

In particular, it is an object of the invention to provide a needle unit for a drug delivery device which enables, or contributes to enable, safe multiple reuse of a skin entering needle portion.

It is another object of the invention to provide such a needle unit which has simple and cost-effective means of enabling establishment of a biostatic environment for the skin entering needle portion.

It is a further object of the invention to provide such a needle unit presenting a reduced risk of degradation of the biostatic environment over time.

It is an even further object of the invention to provide a drug delivery system comprising a drug delivery device and a needle unit which can be reused with the drug delivery device multiple times without entailing an increased risk of microbial contamination.

In the disclosure of the present invention, aspects and embodiments will be described which will address one or more of the above objects and/or which will address objects apparent from the following text.

In a first aspect the invention provides a needle unit according to claim 1.

Hence, a needle unit of the type having a proximal space adapted to accommodate a portion of a variable volume reservoir, such as a drug cartridge, e.g. being embedded in a cartridge holder of a drug delivery device, is provided. The needle unit comprises a needle carrier, a needle tube being fixed to the needle carrier and comprising a distal needle end for providing fluid communication to an injection site, and a needle shield. The needle unit may further comprise coupling means, e.g. comprising a thread and/or a bayonet track, arranged within the proximal space for releasably or non-releasably retaining the variable volume reservoir.

The needle shield carries a sealed chamber configured to accommodate a distal portion of the needle tube. The sealed chamber is sealed distally by a penetrable self-sealing septum, and the needle shield and the needle carrier are capable of relative motion between a first relative position in which the sealed chamber houses the distal needle end, and a second relative position in which the distal needle end protrudes from the sealed chamber through the penetrable self-sealing septum.

The needle unit further comprises a flow channel for establishing fluid communication between the sealed chamber and the proximal space, thereby enabling fluid transfer from an accommodated drug cartridge to the sealed chamber.

The needle tube further comprises a side hole which is a) in fluid communication with the flow channel and b) positioned within the sealed chamber when the needle shield and the needle carrier are in the second relative position.

The side hole may e.g. be produced by drilling or by electrochemical etching. Furthermore, as an example, the needle unit may be designed and dimensioned such that the side hole is positioned in a proximal end portion of the sealed chamber when the needle shield and the needle carrier are in the first relative position and in a distal end portion of the sealed chamber, when the needle shield and the needle carrier are in the second relative position. This will minimise the axial extent of the sealed chamber, while still providing the desired effect thereof. In any case, the side hole may be positioned within the sealed chamber also when the needle shield and the needle carrier are in the first relative position.

In use, when the needle unit is mounted on a drug delivery device, such as e.g. a pen-type injection device, which carries a drug cartridge, and the distal needle end protrudes from the sealed chamber through the self-sealing septum the flow channel and the side hole together provide for fluid communication between the drug cartridge, the sealed chamber and the distal needle end.

Resultantly, when the distal needle end protrudes from the sealed chamber through the self-sealing septum and a dose of drug is expelled from the drug cartridge by operation of the drug delivery device the drug firstly enters the flow channel and is conveyed to the sealed chamber from where part of the dose continues through the needle tube and out of the distal needle end. The sealed chamber, which is sterilised and which contains air prior to use, is thus flushed by, and eventually partly filled with, drug. Hence, after concluded initial dose expelling the sealed chamber contains a combination of drug and air/drug fumes.

This combination of drug and air/drug fumes provides a biostatic environment for the distal portion of the needle tube which keeps it clean until the next injection. During the next injection the drug present in the sealed chamber will be flushed out, or substantially flushed out, and replaced by a new volume of drug from the drug cartridge, and the preservatives in the sealed chamber are thus refreshed, ensuring no or only minimum degradation of the established biostatic environment.

Furthermore, since the side hole allows for filling and flushing of the sealed chamber in connection with the dose expelling itself the need for a separate chamber filling process is avoided, and the needle unit can accordingly be realised with a simple, fixed volume sealed chamber, devoid of any movable walls.

The sealed chamber, and/or any seals thereof, such as a proximal seal having an opening therein for allowing passage of the flow channel, can e.g. be made of butyl rubber or thermoplastic elastomers containing immobilised Zinc (Zi⁺⁺) or immobilised Silver (Ag⁺). These ions are known to inhibit micro-bacterial growth, and by using such elastic materials as seals around e.g. an exterior surface of the flow channel, micro-bacterial contaminants will be neutralised even where the preservative containing drug cannot be brought in permanent contact with such surface.

Non-limiting examples of preservative containing liquid drugs which may be delivered by the drug delivery device and used to establish a biostatic environment in the sealed chamber are blood glucose regulators, such as insulin, insulin analogue, GLP-1, GLP-2, and combinations thereof, as well as growth stimulating hormones. Particularly suitable are for instance the following commercially available drug products: NovoRapid®, NovoLog®, Levemir®, Tresiba®, NovoMix®, Ryzodeg®, Xultophy®, Victoza®, Saxenda®, Ozempic® and Norditropin®.

The sealed chamber is sufficiently large to accommodate the distal portion of the needle tube, yet sufficiently small to provide for the flow properties described above and to enable establishment of a satisfactory microbe-hostile environment which prevents contamination even though the distal portion of the needle tube may not be fully submerged in the liquid drug.

The inventors have identified that if the sealed chamber has a volume in the range [5 μL; 60 μL] a particularly attractive solution satisfying desires and requirements to, respectively, needle unit size and dose accuracy of the drug delivery device/needle unit system is obtained. For example, the sealed chamber may have a volume in the range [7 μL; 20 μL] which appears to be an optimum compromise between cleaning capacity and loss of drug during the first dose expelling, where a portion of the expelled dose is deposited in the sealed chamber.

During subsequent doses, little or no drug is lost, as the sealed chamber is being flushed, or substantially flushed, by the new dose. Since the first dose expelling should preferably be an air-shot the loss of drug to the sealed chamber will have no physical impact on the user. However, with a volume in the range [7 μL; 20 μL], even if the first dose expelling is not an air-shot but instead a regular dose administration the lost drug volume is well below authority accepted dose inaccuracy thresholds for systems delivering a highly potent drug as insulin, and the loss will accordingly not have any noticeable effect on the user.

The sealed chamber may e.g. be cylindrical and may e.g. have an inner diameter in the range [0.5 mm; 2.5 mm].

The flow channel may comprise a proximal needle end configured to penetrate a reservoir septum, such as e.g. a drug cartridge septum. This will allow for a simple and inexpensive establishment of fluid communication between the sealed chamber and the variable volume reservoir, e.g. the drug cartridge, when the latter is introduced into the proximal space of the needle unit.

The flow channel may constitute a proximal portion of the needle tube, being fluidly connected with the distal portion. Thereby, fluid communication between the variable volume reservoir, the sealed chamber and the injection site will be established via a single needle tube which enables a particularly slim construction of the sealed chamber and thereby of the needle unit.

Alternatively, the flow channel may comprise a separate, second needle tube extending between the proximal space and the sealed chamber. The second needle tube may e.g. be arranged in parallel with the needle tube. The liquid drug is thus conveyed from the variable volume reservoir to the sealed chamber via the second needle tube, and will at some point, after having partially, e.g. substantially fully, filled the sealed chamber, enter the needle tube through the side hole and flow on to the distal needle end. In this case the second needle tube, part of which residing in the variable volume reservoir, may be refrained from exposure to the surroundings. The second needle tube may be slidably received in the proximal seal of the sealed chamber, allowing relative axial motion between the sealed chamber and the second needle tube.

The fill level of the sealed chamber may be optimised by various means, e.g. involving a particular construction of the sealed chamber, and/or a particular form of the side hole, to provoke a desired direction of drug inflow from the flow channel into the sealed chamber, and/or a particular placement of the side hole in the needle tube.

In one example thereof the sealed chamber comprises a proximal cylindrical zone, a distal cylindrical zone, and an intermediate conical zone tapering towards the distal cylindrical zone, and the side hole is positioned in the distal cylindrical zone when the needle shield and the needle carrier are in the second relative position. Drug flowing from the flow channel into the sealed chamber will thereby enter the distal cylindrical zone and from there flow towards the larger volume at the proximal cylindrical zone, displacing the air to the distal cylindrical zone from which most of it will escape through the side hole.

A similar effect may be achieved by providing a side hole which is positioned in a distal portion of the sealed chamber when the needle shield and the needle carrier are in the second relative position and which has a sloping edge portion connecting an interior surface of the needle tube with an exterior surface of the needle tube, where the sloping edge portion is sloped in the proximal direction from the interior surface of the needle tube to the exterior surface of the needle tube, as this provokes a proximal inflow of drug from the flow channel into the sealed chamber. An asymmetric side hole like that may be produced by drilling through a side wall portion of the needle tube at an angle different from 90°, e.g. at an angle of approximately 45°.

The placement of the side hole, as well as the number of side holes, in the sealed chamber at the time of filling are other factors which may be utilised to control the fill level. For example, the needle tube may comprise a second side hole, and the side hole and the second side hole may be arranged such that the side hole is positioned in a distal portion, such as in a distal end portion, of the sealed chamber and the second side hole is positioned in a proximal portion, such as in a proximal end portion, of the sealed chamber, when the needle shield and the needle carrier are in the second relative position. Due to the flow resistance in the needle tube this will lead most of the pressurised drug flowing through the flow channel directly into the sealed chamber through the second side hole at the proximal portion of the sealed chamber, and as the sealed chamber is increasingly filled with liquid from the proximal portion air is expelled through the side hole in the distal portion, eventually leaving only a small volume of air in the distal portion of the sealed chamber.

In particular examples, the needle tube comprises a blocking structure arranged between the side hole and the second side hole, e.g. extending from the side hole to the second side hole, to ensure that the liquid conveyed through the flow channel is forced to enter the sealed chamber before eventually leaving the distal needle end. This will enable a practically complete de-aeration of the sealed chamber and furthermore provide for a full, or substantially full, exchange of drug in the sealed chamber at each next injection action, according to the first-in-first-out principle.

Naturally, the various means for optimising the fill level of the sealed chamber may be combined in any appropriate manner.

The sealed chamber is fixedly arranged within the needle shield, e.g. at a distal end portion thereof. The needle shield may be biased, e.g. by a spring member, towards the first relative position with respect to the needle carrier. The needle shield will thus automatically cover, and the sealed chamber will automatically accommodate, the distal portion of the needle tube, i.e. the portion of the needle tube configured for insertion into the user, both before and after a dose expelling event, when the needle unit is not pressed against the skin.

The needle unit may further comprise a needle support arranged between the sealed chamber and the needle carrier, where the needle support is configured to slidably receive at least one needle tube. Such needle support may resist bending of any needle tube present in the needle unit.

In a second aspect of the invention a needle unit for use with a drug delivery device is provided, the needle unit comprising a needle hub, a needle tube being fixed to the needle hub and comprising a proximal needle end for providing fluid communication with a drug reservoir and a distal needle end for providing fluid communication with an injection site, and a needle shield carrying a sealed chamber, the sealed chamber being sealed distally by a penetrable self-sealing septum, wherein the needle shield and the needle hub are capable of relative motion between an extended relative position in which the sealed chamber houses the distal needle end and a first wall portion of the needle tube, and a retracted relative position in which the distal needle end protrudes from the sealed chamber through the penetrable self-sealing septum and the sealed chamber houses a second wall portion of the needle tube, and wherein the second wall portion of the needle tube comprises a side hole providing fluid communication between the proximal needle end and the sealed chamber.

Thereby, a needle unit may be provided which employs a needle tube as conventionally used in pen-needle type of injection needle units, albeit with a side hole arranged therein. The first wall portion and the second wall portion may overlap, and the side hole may be arranged in a portion of the needle tube which forms part of both the first wall portion and the second wall portion, whereby the side hole is positioned within the sealed chamber at all times, i.e. both before use, during use and between uses. This enables filling of the sealed chamber both when the needle shield and the needle hub are in the extended relative position and in the retracted relative position.

In a third aspect of the invention an injection system comprising an injection device and a needle unit as described in the above is provided. The injection device holds, or is adapted to hold, a preservative containing liquid drug. The needle unit may comprise first coupling means, e.g. comprising a thread and/or a bayonet track, arranged within the proximal space, and the injection device may comprise second coupling means for mating connection with the first coupling means.

In a fourth aspect of the invention a method of enabling establishment of a biostatic environment for an injection needle having a distal needle end and a side hole is provided. The method comprises i) fixing the injection needle to a needle carrier and providing fluid communication between the side hole and a receiving space capable of receiving a drug reservoir holding a preservative containing liquid, ii) providing a sealed chamber for accommodating a distal portion of the injection needle, wherein the sealed chamber a) is sterilised and sealed distally by a penetrable septum, and b) has a volume in the range [5 μL; 60 μL], and iii) arranging the sealed chamber and the needle carrier such that the sealed chamber and the needle carrier are capable of relative motion between a first relative position in which the sealed chamber houses the distal needle end and a second relative position in which the distal needle end protrudes from the sealed chamber through the penetrable self-sealing septum, and such that the side hole is positioned within the sealed chamber when the sealed chamber and the needle carrier are in the second relative position.

By this method the distal needle end is housed in the sealed chamber between injection actions, which sealed chamber is at least partially filled with preservative containing liquid from the drug reservoir by an initial user action and subsequently, during each injection, flushed and substantially replaced by another volume of the preservative containing liquid, thereby renewing the micro-bacterial growth inhibitors in, and maintaining the cleaning capacity of, the sealed chamber.

Hence, the biostatic environment can be established simply by expelling a dose of the preservative containing liquid from the drug reservoir through the distal needle end, as thereby a volume of the drug will remain in the sealed chamber, this volume being sufficient to allow the preservatives in the liquid drug and in the drug fumes to produce and uphold a conserved space. As the introduction of preservative containing liquid is an automatic and unavoidable consequence of any user induced dose expelling action the preservatives in the sealed chamber will be renewed every time a dose is administered, and the maintenance of the biostatic environment is thus independent of dedicated operational steps which a user may neglect.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a perspective view of an injection device with a needle unit according to an embodiment of the invention attached thereto,

FIG. 2 is a longitudinal section view of the needle unit shown in FIG. 1,

FIGS. 3 and 4 are perspective views of components of an exemplary biostatic chamber for the injection needle,

FIG. 5 is a longitudinal section view of the needle unit with an attached drug cartridge,

FIG. 6 depicts the needle unit in various states during use,

FIG. 7 is a longitudinally sectioned perspective view of the biostatic chamber and the injection needle, respectively in a storage state and in a use state,

FIG. 8 shows the biostatic chamber and the injection needle in various states during use,

FIG. 9 shows a biostatic chamber and an injection needle as used in a second embodiment of the invention in various states during use,

FIG. 10 shows a biostatic chamber and an injection needle as used in a third embodiment of the invention in various states during use,

FIG. 11 is a longitudinal section view of a needle unit according to a fourth embodiment of the invention with a drug cartridge attached thereto and in various states during use,

FIG. 12 is a longitudinal section view of a needle unit according to a fifth embodiment of the invention with a drug cartridge attached thereto and in various states during use,

FIG. 13 is an exploded, partly longitudinally sectioned, view of a sub-assembly for use in a needle unit according to a sixth embodiment of the invention,

FIG. 14 is a longitudinal section view of the needle unit incorporating the sub-assembly of FIG. 13, and

FIG. 15 is a longitudinal section view of the sub-assembly in various states during use of the needle unit.

In the figures like structures are mainly identified by like reference numerals.

### DESCRIPTION OF EXEMPLARY EMBODIMENTS

When in the following relative expressions, such as “upwards” and “downwards” and “left” and “right”, are used, these refer to the appended figures and not necessarily to an actual situation of use. The shown figures are schematic representations for which reason the configuration of the different structures as well as their relative dimensions are intended to serve illustrative purposes only.

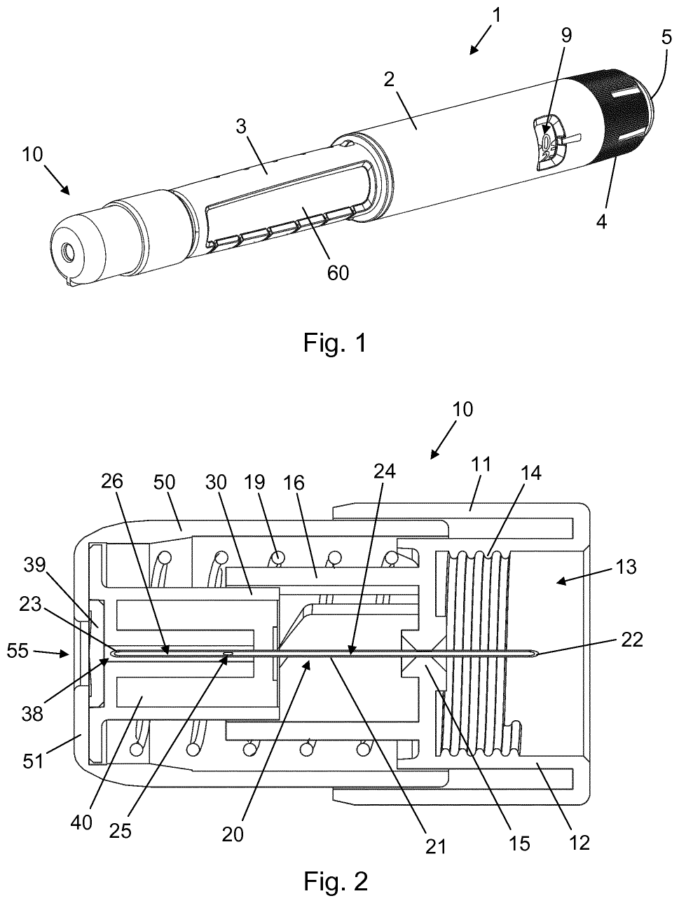

FIG. 1 is a perspective view of an injection pen 1 having a needle unit 10 according to an exemplary embodiment of the invention attached thereto. The injection pen 1 has a longitudinal housing 2 which accommodates an injection mechanism (not visible). A cartridge holder 3 is attached to the distal end of the housing 2 and supports a cartridge 60 holding a liquid drug. In the present case the cartridge holder 3 is fixedly attached to the housing 2, as the injection pen 1 is of the so-called prefilled injection device type. However, in other cases the cartridge holder 3 could be detachably attached. In conventional fashion the injection pen 1 has a dose dial button 4 for selective setting of a dose to be delivered and an injection button 5 for actuation of the injection mechanism arranged at its proximal end portion, and a currently set dose can be viewed through a window 9 in the housing 2. Non-exhaustive examples of injection devices which may be used with any of the needle units presented herein are FlexTouch® and FlexPen®, manufactured by Novo Nordisk A/S.

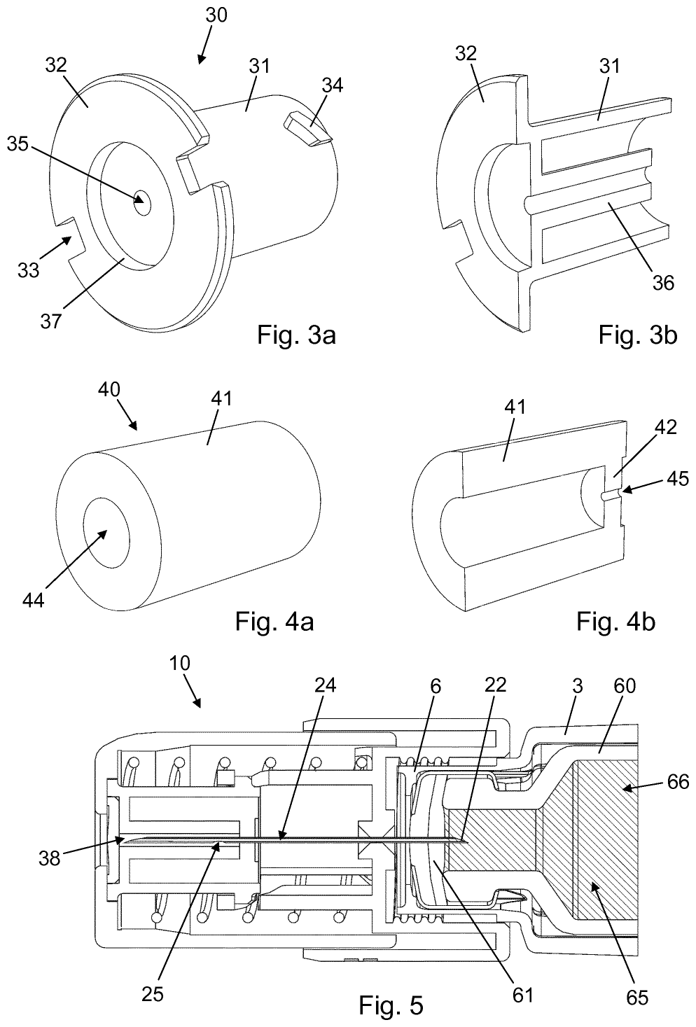

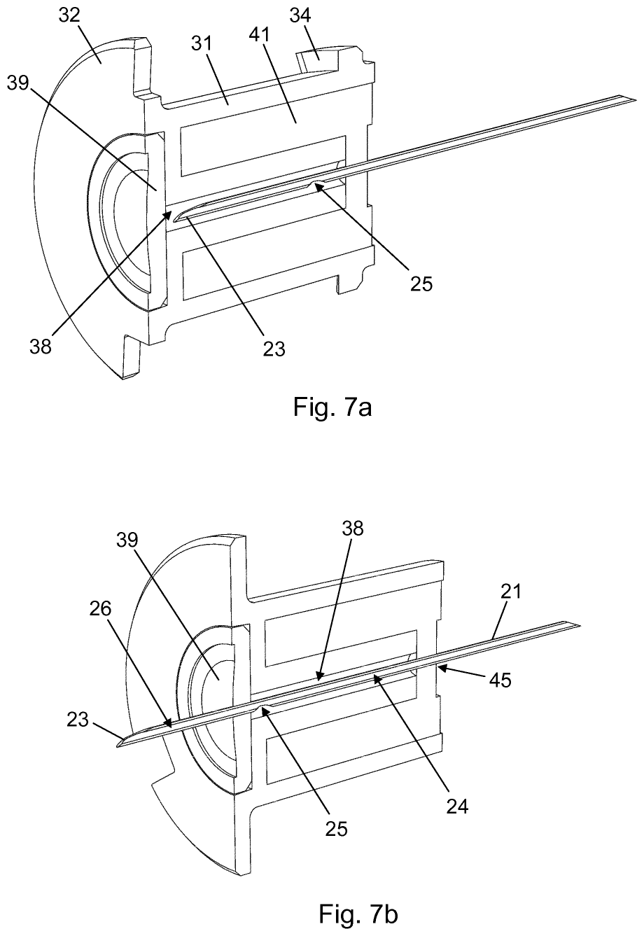

FIG. 2 is a longitudinal section view of the needle unit 10 in a pre-use state. The needle unit 10 has a base member 11 comprising an inner section 12, which defines a proximal space 13 and carries a thread 14 for reception of a needle mount 6 (ref. FIG. 5) of the injection pen 1, a needle hub 15, which carries an injection needle 20 extending along a general longitudinal axis, and an axial guide 16. The needle unit 10 further comprises a needle shield 50 which carries a biostatic chamber sub-assembly formed by a chamber structure 30, a sealing sleeve 40, and a self-sealing chamber septum 39, together defining a sealed chamber 38. The sealing sleeve 40 is made of a thermoplastic elastomer and contains immobilised Silver (Ag⁺) to neutralise micro-bacterial contaminants.

The needle shield 50 and the base member 11 are capable of relative axial motion during which the chamber structure 30 will be guided by the axial guide 16. The needle shield 50 comprises a transversal contact face 51 which is configured for abutment with a skin section (not shown) of a user and which has an orifice 55 therein.

The injection needle 20 comprises an elongated needle tube 21, which is fixedly mounted in the needle hub 15, a proximal needle end 22 which is configured for penetration of a self-sealing cartridge septum 61 (ref. FIG. 5) and entry into a cartridge interior 65 (ref. FIG. 5), and a distal needle end 23 configured for insertion through the skin of the user.

In the depicted pre-use state of the needle unit 10 the injection needle 20 extends through the sealing sleeve 40 and a distal portion of the needle tube 21, including the distal needle end 23, resides within the sealed chamber 38 which is otherwise filled with air. The needle tube 21 is provided with a side hole 25, dividing the lumen of the needle tube 21 into a proximal flow channel 24, leading from the proximal needle end 22 to the side hole 25, and a distal flow channel 26, leading from the side hole 25 to the distal needle end 23.

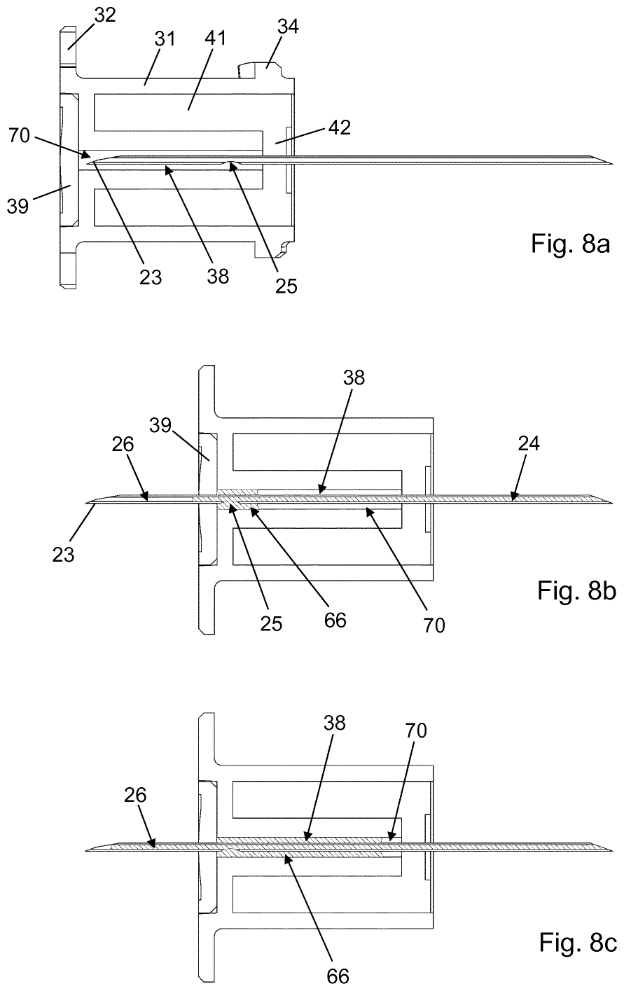

The needle shield 50 and the base member 11 are capable of relative axial motion between an extended relative position (e.g. as shown in FIG. 2) in which both the distal needle end 23 and the side hole 25 are accommodated within the sealed chamber 38 and a retracted relative position (e.g. as shown in FIG. 6c) in which the distal needle end 23 protrudes through the chamber septum 39 and the orifice 55 and the side hole 25 is accommodated within the sealed chamber 38. The needle shield 50 and the base member 11 are biased towards the .extended relative position by a compression spring 19.

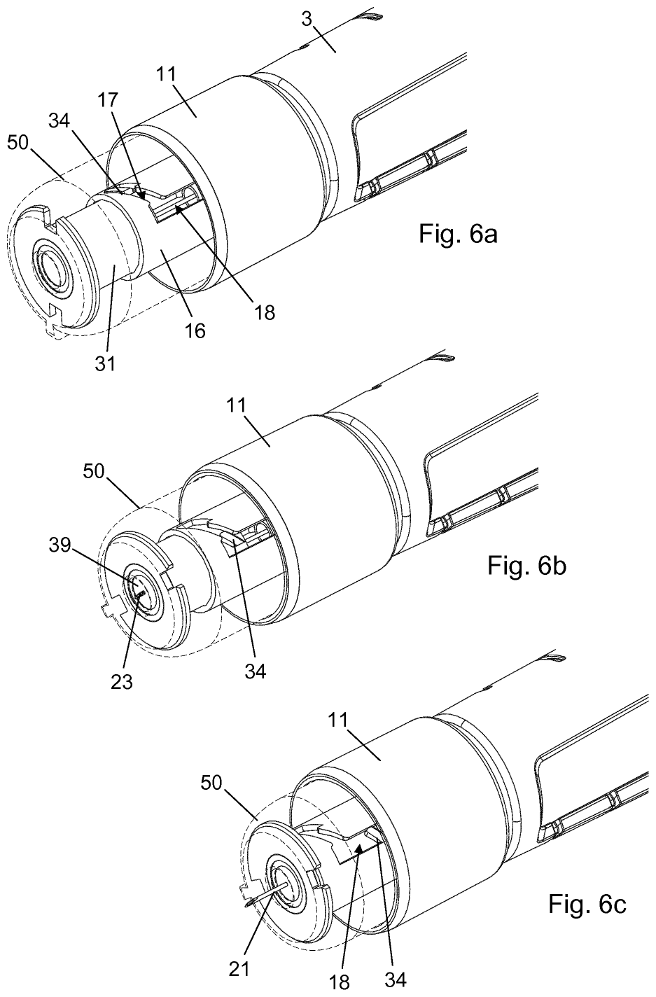

FIG. 3a is a perspective view of the chamber structure 30, which comprises an outer cylindrical wall 31, with a protrusion 34 on a proximal end portion thereof, and a distal flange 32. The flange 32 is of circular shape and has a pair of notches 33 arranged diametrically opposite one another, enabling a rotational fixation of the chamber structure 30 to the needle shield 50. The flange 32 extends axially beyond the outer cylindrical wall 31, thereby defining a recess 37 for accommodation of the chamber septum 39. As seen in FIG. 2 the chamber septum 39 is sandwiched between the chamber structure 30 and an interior portion of the transversal contact face 51.

FIG. 3b is a longitudinally sectioned perspective view of the chamber structure 30 showing an inner cylindrical wall 36 which forms a side wall of the sealed chamber 38. A distal end of the inner cylindrical wall 36 forms an opening 35 (ref. FIG. 3a) through which the distal needle end 23 escapes the sealed chamber 38 during use of the needle unit 10.

FIGS. 4a and 4b are, respectively, a perspective view and a longitudinally sectioned perspective view of the sealing sleeve 40. The sealing sleeve 40 comprises a cylindrical body 41 having a wall thickness and a lumen 44 which are adapted for reception between the inner cylindrical wall 36 and the outer cylindrical wall 31 of the chamber structure 30. The sealing sleeve 40 has a proximal end wall 42 with a through-going central bore 45 dimensioned for slidable sealing connection with the needle tube 21. In the needle unit 10 the sealing sleeve 40 fits tightly around the inner cylindrical wall 36, thereby providing a rear, or proximal, seal for the sealed chamber 38, the chamber septum 39 providing a front, or distal, seal.

FIG. 5 shows the needle unit 10 where the proximal space 13 is occupied by a head portion of the cartridge 60 and the needle mount 6 of the cartridge holder 3, the latter being mated with the thread 14. In this position of the cartridge holder 3 the proximal needle end 22 has penetrated the cartridge septum 61, and a proximal end portion of the needle tube 21 extends into the cartridge interior 65, establishing fluid communication with a preservative containing liquid drug 66 therein.

FIGS. 6a-c show three different states of the needle unit 10, where FIG. 6a illustrates a preuse, locked state, FIG. 6b illustrates a (near) priming state, and FIG. 6c illustrates a drug expelling state. The guide 16 is provided with two connected tracks, an inclined track 17 and an axial track 18. In the locked state the protrusion 34 is parked in the inclined track 17, preventing proximal translational motion of the needle shield 50 relative to the base member 11. This reflects the extended relative position of the needle shield 50 and the base member 11. In the locked state of the needle unit 10 the user cannot inadvertently push back the needle shield 50 and is therefore not in risk of accidental needle stick injuries.

If the user rotates the needle shield 50 clockwise relative to the base member 11 the outer cylindrical wall 31 of the chamber structure 30 will rotate accordingly due to the rotationally interlocked relationship between the needle shield 50 and the flange 32. This will cause the protrusion 34 to travel the inclined track 17 which will consequently cause the needle shield 50 to move a short distance proximally relative to the base member 11 due to the axially interlocked relationship between the needle shield 50 and the chamber structure 30. As a result of the proximal movement of the needle shield 50 the distal needle end 23 penetrates the chamber septum 39 and extends slightly through the orifice 55. This reflects a priming position of the needle shield 50 and is illustrated in FIG. 6b (actually, FIG. 6b shows the needle unit 10 just before the protrusion 34 reaches the end of the inclined track 17 and therefore just before the needle shield 50 reaches the priming position).

When the protrusion 34 is at the end of the inclined track 17 it enters the axial track 18. At this point the needle shield 50 is translationally unlocked and can be pressed proximally relative to the base member 11, e.g. by placing the contact face 51 on the skin and pressing the cartridge holder 3 towards the skin, to expose the distal end portion of the needle tube 21. This reflects a dose expelling position of the needle shield 50 and is illustrated in FIG. 6c.

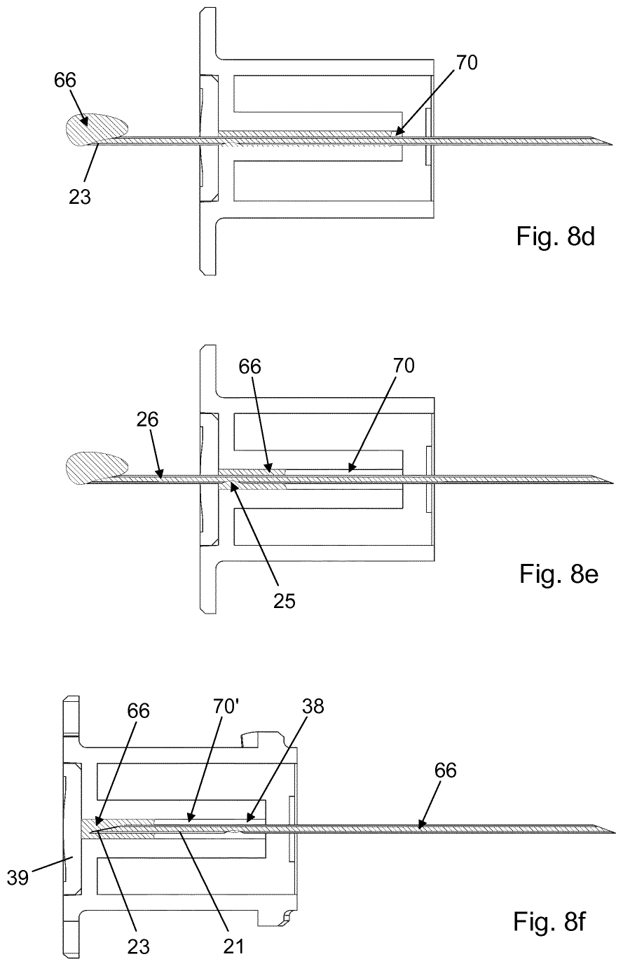

FIGS. 7a and 7b are longitudinally sectioned perspective views of the injection needle 20 and the biostatic chamber sub-assembly constituted by the chamber structure 30, the sealing sleeve 40 and the chamber septum 39, as mutually positioned in the extended relative position, respectively the retracted relative position of the needle shield 50 and the base member 11. In the former of these relative positions the side hole 25 is positioned in a proximal portion of the sealed chamber 38, whereas in the latter of the relative positions the side hole 25 is positioned in a distal portion of the sealed chamber 38.

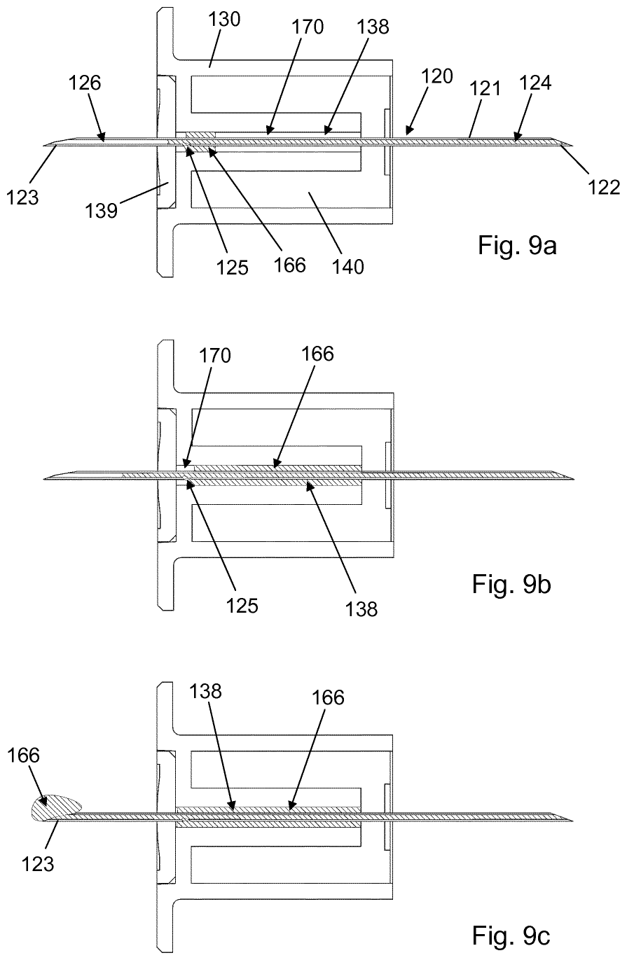

FIGS. 8a-8f show the principle of the establishment of a biostatic environment for the distal portion of the needle tube 21 which goes into the user during dose expelling events. In connection with a first use of the injection pen 1 the user may prepare for a small dose to be expelled in order to prime the needle unit 10. However, the user may neglect to perform this priming and instead immediately prepare for administration of a proper dose. Regardless of which the principle is the same and will in the following be described with respect to the situation, where the user administers a proper dose as the very first action. For the sake of clarity only the injection needle 20 and the biostatic chamber sub-assembly are depicted in these figures.

Having prepared the desired dose by operation of the dose dial button 4 the user places the contact face 51 on the skin surface at the chosen injection site and presses the housing 2 against the skin. This brings the needle shield 50 and the base member 11 from the extended relative position (FIG. 8a) to the retracted relative position (FIG. 8b), against the force of the compression spring 19. Depression of the injection button 5 leads to a pressurisation of the liquid drug 66 in the cartridge 60 which then begins to flow through the proximal needle end 22 and into the proximal flow channel 24.

When the liquid drug 66 reaches the side hole 25 it will flow into the sealed chamber 38, compressing existing air 70 therein (FIG. 8b). As more liquid drug 66 is pressed out of the cartridge 60 the sealed chamber 38 is gradually filled until the air 70 cannot be compressed further. At this point the liquid drug 66 leaving the cartridge 60 will flow straight through the proximal flow channel 24, and into the distal flow channel 26, bypassing the side hole 25 due to the hydraulic pressure in the sealed chamber 38 (FIG. 8c). Being pushed through the distal flow channel 26 by the pressure gradient the liquid drug 66 finally reaches the distal needle end 23 and enters the subcutaneous tissue of the user (not shown) (FIG. 8d).

At the end stages of the injection action, when the pressure in the needle lumen decreases, the air 70 will expand and press some of the liquid drug 66 present in the sealed chamber 38 back through the side hole 25 (FIG. 8e). Eventually, when the injection procedure is over and the user removes the injection needle 20 from the skin by pulling back the injection pen 1 the compression spring 19 automatically brings the needle shield 50 and the base member 11 into the extended relative position where the distal needle end 23 is accommodated in the sealed chamber 38. FIG. 8f illustrates the resulting between-use storage condition of the distal portion of the needle tube 21. It is seen that the interior of the needle tube 21 is filled with liquid drug 66 and that the exterior of the distal portion of the needle tube 21, which gets in contact with the users skin, is stored in a combination of the liquid drug 66 remaining in the sealed chamber 38 and drug fumes 70′. The preservatives in the liquid drug 66 and in the drug fumes 70′ inhibit micro-bacterial growth and a biostatic environment is thus established for the lumen as well as for the exterior surface of the distal portion of the needle tube 21.

When during each subsequent injection action a new dose is prepared and expelled from the cartridge 60 a fresh volume of liquid drug 66 will enter the sealed chamber 38 through the side hole 25, compressing the drug fumes 70′ and mixing with the liquid drug 66 already present therein. At the end of such injection action, when the drug fumes 70′ has again expanded, the liquid drug 66 remaining in the sealed chamber will be at least partly renewed, this reducing the risk of potential degradation over time of the biostatic environment due to diffusion of preservatives from the sealed chamber 38.

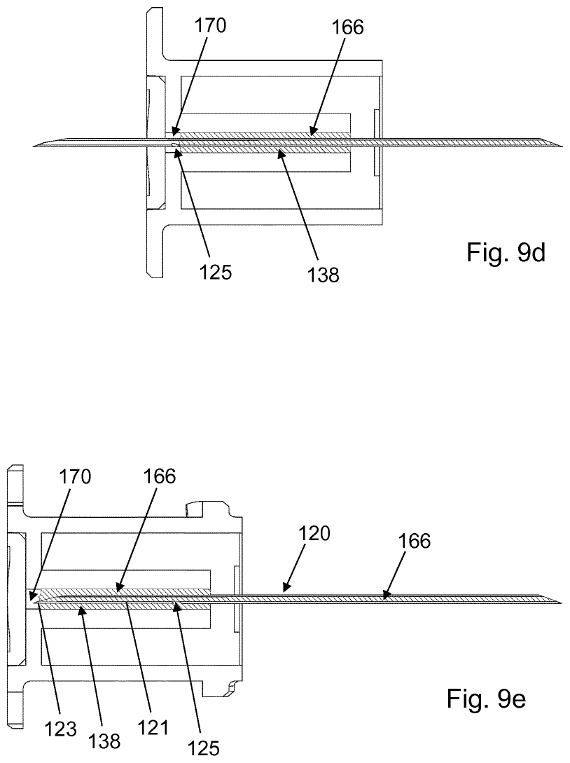

FIGS. 9a-e show a biostatic chamber sub-assembly and an injection needle 120 as used in a needle unit according to a second embodiment of the invention. All parts of the needle unit according to the second embodiment are identical to those of the first embodiment, except from the injection needle 120. Hence, the biostatic chamber sub-assembly comprises, similarly to the previous biostatic chamber sub-assembly, a chamber structure 130, a sealing sleeve 140, and a self-sealing chamber septum 139, together defining a cylindrical sealed chamber 138. Also, the injection needle 120, albeit being different, does comprise a needle tube 121 having a proximal needle end 122 and a distal needle end 123 just as the injection needle 20 of the former embodiment.

The needle tube 121 comprises a side hole 125 dividing its lumen into a proximal flow channel 124, leading from the proximal needle end 122 to the side hole 125, and a distal flow channel 126, leading from the side hole 125 to the distal needle end 123. The side hole 125 is bored at an angle of approximately 45° from a perpendicular bore direction, and the resulting edge portion connecting the interior surface with the exterior surface of the needle tube 121 is sloped approximately 45° towards the proximal end of the sealed chamber 138. Liquid drug 166 flowing from a drug reservoir through the proximal flow channel 124 will by the shape of the side hole 125 be forced into the sealed chamber 138 in a rearwards, or proximal, flow direction (FIG. 9a). As a result air 170 present in the sealed chamber 138 will be forced rearwards in a swirling motion and will eventually exit through the side hole 125 as bubbles in the liquid drug 166 due to the turbulent flow (FIG. 9b), leaving only a small volume of compressed air 170 at the front of the sealed chamber 138, while the injection itself takes place, i.e. while the liquid drug 166 pours through the distal flow channel 126 and out of the distal needle end 123 (FIG. 9c).

At the end stage of the injection when the hydraulic pressure drops and the compressed air 170 expands some of the liquid drug 166 is expelled out through the side hole 125 and an uncompressed volume of air 170 thus remains in the sealed chamber 138, which is at this point primarily filled with liquid drug 166 (FIG. 9d). A subsequent retraction of the injection needle 120 from the skin brings the distal portion of the needle tube 121 to an accommodated position within the sealed chamber 138, in a manner similar to the above described, and that portion of the needle tube 121 is thus almost completely submerged in the preservative containing liquid drug 166 in the between-use period with only a small volume of drug fumes 170′ being present as well. A biostatic environment is thus established for the lumen as well as for the exterior surface of the distal portion of the needle tube 121, and as with the previous embodiment any subsequent injection action will flush the sealed chamber 138 and at least partly renew the liquid drug 166 therein, maintaining an adequate level of microbacterial growth inhibiting substance.

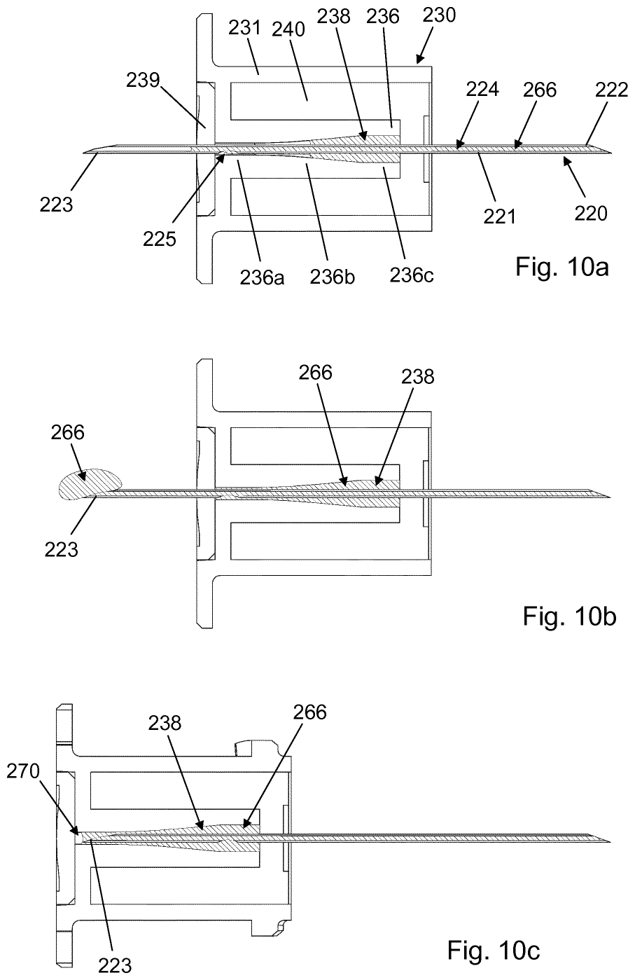

FIGS. 10a-10c show a biostatic chamber sub-assembly and an injection needle 220 as used in a needle unit according to a third embodiment of the invention. All parts of the needle unit according to the third embodiment are identical to those of the first embodiment, except from a part of the biostatic chamber sub-assembly. Hence, the injection needle 220 comprises a needle tube 221 having a proximal needle end 222, a distal needle end 223, and a side hole 225 which is symmetric about an axis perpendicular to the longitudinal axis of the needle tube 221. The side hole 225 divides the lumen of the injection needle 20 into a proximal flow path 220 leading from the proximal needle end 222 to the side hole 225, and a distal flow channel 226, leading from the side hole 225 to the distal needle end 223. The biostatic chamber sub-assembly comprises a chamber structure 230, a sealing sleeve 240, and a self-sealing chamber septum 239, together defining a sealed chamber 238.

The chamber structure 230 has an outer cylindrical wall 231, identical to the outer cylindrical wall 31 of the first embodiment, and an inner wall 236. The inner wall 236 has a cylindrical exterior surface around which the sealing sleeve 240 is fitted, in a manner similar to the above described, but is of varying thickness, which provides a sealed chamber 238 having a distal cylindrical zone 236a of a first diameter, a proximal cylindrical zone 236c of a second diameter, being larger than the first diameter, and an intermediate conical zone 236b bridging the distal cylindrical zone 236a and the proximal cylindrical zone 236c.

This third embodiment presents an alternative way of obtaining substantially the same fill level of the sealed chamber 238 as that of the sealed chamber 138 according to the second embodiment. The effect of the three chamber zones is similar to that of the skewed side hole 125, i.e. liquid drug 266 flowing through the proximal flow channel 224 enters the side hole 225 and is forced rearwards in the sealed chamber 238 towards the larger proximal cylindrical3 zone 236c, whereby air present in the sealed chamber is urged distally and eventually exits the side hole 225 as air bubbles in the liquid drug 266 (FIG. 10a).

As the injection progresses the sealed chamber 238 becomes almost completely filled with liquid drug 266 (FIG. 10b) and at the end only a small volume of air remains in the sealed chamber 238. In the between-use periods the distal portion of the needle tube 221 is thus almost completely submerged in preservative containing liquid drug 266 with only a small volume of drug fumes 270′ being present as well.

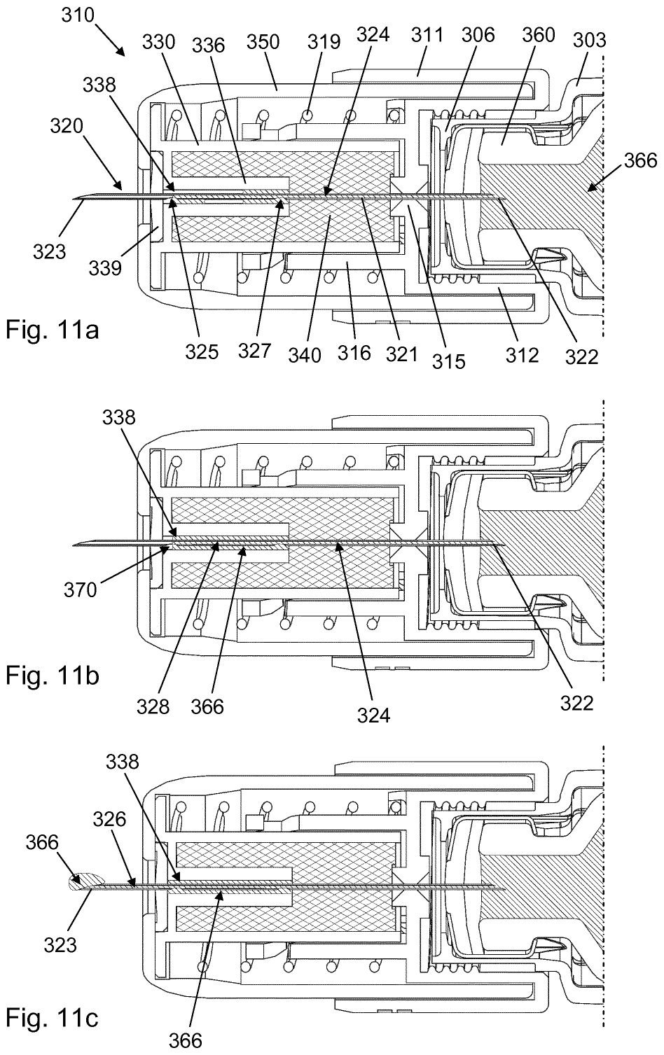

FIGS. 11a-11e are longitudinal section views of a needle unit 310 according to a fourth embodiment of the invention attached to a needle mount 306 of a cartridge holder 303 which forms part of an injection device, e.g. of the type previously described. The needle unit 310 has a base member 311 which is largely similar to the base member 11 described in connection with the first embodiment of the invention, i.e. comprising an inner section 312, which defines a proximal space formed for reception of the needle mount 306, a needle hub 315 which carries an injection needle 320, and an axially extending guide 316. The needle unit 310 further comprises a needle shield 350 which carries a biostatic chamber subassembly formed by a chamber structure 330, a sealing sleeve 340, and a self-sealing chamber septum 339, together defining a sealed chamber 338. The sealing sleeve 340 is made of a thermoplastic elastomer and contains immobilised Zinc (Zi⁺⁺) to neutralise microbacterial contaminants.

The needle shield 350 and the base member 311 are capable of relative axial motion during which the chamber structure 330 will be guided by the guide 316, in the same manner described in connection with the first embodiment of the invention.

The injection needle 320 comprises an elongated needle tube 321, which is fixedly mounted in the needle hub 315, a proximal needle end 322 which is configured for penetration of a self-sealing cartridge septum and entry into a cartridge interior, thereby establishing fluid communication with a liquid drug 366, and a distal needle end 323 configured for insertion through the skin of the user.

In a pre-use state of the needle unit 310 the injection needle 320 extends through the sealing sleeve 340 and a distal portion of the needle tube 321, including the distal needle end 323, resides within the sealed chamber 338 which is otherwise filled with air. The needle tube 321 is provided with a distal side hole 325 and a proximal side hole 327, dividing the lumen of the needle tube 321 into a proximal flow channel 324, leading from the proximal needle end 322 to the proximal side hole 327, an intermediate flow channel 328, leading from the proximal side hole 327 to the distal side hole 325, and a distal flow channel 326, leading from the distal side hole 325 to the distal needle end 323. The axial distance between the distal side hole 325 and the proximal side hole 327 is correlated with the axial dimension of the sealed chamber 338 such that in at least one particular position of the needle tube 321 relative to the chamber structure 330 the distal side hole 325 is positioned in a distal end portion and the proximal side hole 327 is positioned in a proximal end portion of the sealed chamber 338.

The needle shield 350 and the base member 311 are capable of relative axial motion between an extended relative position (e.g. as shown in FIG. 11e) in which both the distal needle end 323 and the distal side hole 325 are accommodated within the sealed chamber 338 and a retracted relative position (e.g. as shown in FIG. 11a) in which the distal needle end 323 protrudes through the chamber septum 339 and the distal side hole 325 as well as the proximal side hole 327 are accommodated within the sealed chamber 338. The needle shield 350 and the base member 311 are biased towards the extended relative position by a compression spring 319.

The relative motion between the needle shield 350 and the base member 311 is enabled by means identical to the ones described in connection with the first embodiment of the invention. Consequently, a detailed description of this relative motion in connection with the present embodiment will be omitted.

FIG. 11a shows the needle unit 310 in an initial state during a very first drug injection event. The user has placed the needle unit 310 against the skin and pressed the injection device towards the skin to thereby bring the needle shield 350 and the base member 311 to the retracted relative position in which the distal needle end 323 protrudes through the chamber septum 339 and resides in a subcutaneous compartment within the user (not shown). As the cartridge 360 becomes pressurised and liquid drug 366 resultantly flows through the proximal flow channel 324 the proximal side hole 327 will allow entry of the liquid drug 366 into a proximal end portion of the sealed chamber 338. In fact, the flow resistance in the needle tube 321 will primarily motivate drug flow into the sealed chamber 338 rather than into the intermediate flow channel 328, and since the liquid drug 366 thus initiates a filling of the sealed chamber 338 from the proximal end portion thereof the air 370 originally present in the sealed chamber 338 is gradually forced out through the distal side hole 325 (FIG. 11b), whereby the sealed chamber 338 becomes almost completely de-aerated as the liquid drug 366 continues out through the distal side hole 325, into the distal flow channel 326 and through the distal needle end 323 (FIG. 11c).

At the end stages of the injection action, when the pressure in the needle lumen decreases, the small volume of air 370 present in the sealed chamber 338 will expand (FIG. 11d), and when the injection needle 320 is withdrawn from the skin and the needle shield 350 and the base member 311 returns to the extended relative position (FIG. 11e) the distal portion of the needle tube 321 is submerged in preservative containing liquid drug 366 which together with drug fumes 370′ will provide a biostatic environment for this portion of the needle tube 321 in between-use periods. As with the previous embodiment any subsequent injection action will flush the sealed chamber 338 and at least partly renew the liquid drug 366 therein, maintaining an adequate level of micro-bacterial growth inhibiting substance.

In the between-use state of the needle unit 310 the distal side hole 325 is positioned within the sealed chamber 338, whereas the proximal side hole 327 and the majority of the portion of the needle tube 321 that defines the intermediate flow channel 328 are positioned in the sealing sleeve 340. However, the immobilised Zinc (Zi⁺⁺) in the sealing sleeve 340 will provide for neutralisation of potential micro-bacterial contaminants in that area even though the preservative containing drug 366 is not in permanent contact therewith.

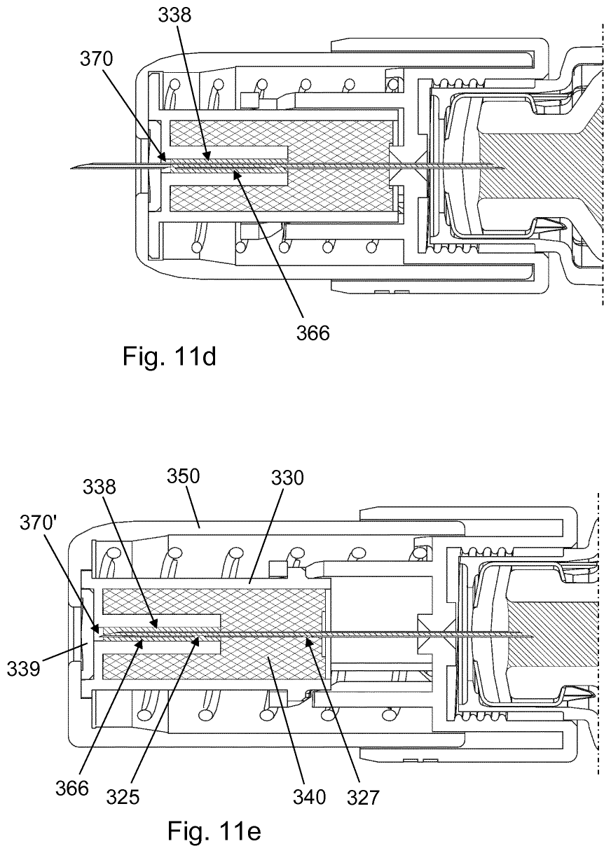

FIGS. 12a-12c are longitudinal section views of a needle unit 410 according to a fifth embodiment of the invention attached to a needle mount 406 of a cartridge holder 403 which forms part of an injection device, e.g. of the type previously described. The needle unit 410 is a variation of the needle unit 310 described above in connection with the fourth embodiment of the invention. It has a base member 411 comprising an inner section 412, which defines a proximal space formed for reception of the needle mount 406, a needle hub 415 which carries an injection needle 420, and an axially extending guide 416. The needle unit 410 further comprises a needle shield 450 which carries a biostatic chamber sub-assembly formed by a chamber structure 430, a sealing sleeve 440, and a self-sealing chamber septum 439, together defining a sealed chamber 438. The sealing sleeve 440 is made of a thermoplastic elastomer and contains immobilised Zinc (Zi⁺⁺) to neutralise micro-bacterial contaminants.

The needle shield 450 and the base member 411 are capable of relative axial motion during which the chamber structure 430 will be guided by the guide 416, in the same manner described in connection with the first embodiment of the invention.

The injection needle 420 comprises an elongated needle tube 421, which is fixedly mounted in the needle hub 415, a proximal needle end 422 which is configured for penetration of a self-sealing cartridge septum and entry into a cartridge interior, thereby establishing fluid communication with a liquid drug 466, and a distal needle end 423 configured for insertion through the skin of the user.

In a pre-use state of the needle unit 410 the injection needle 420 extends through the sealing sleeve 440 and a distal portion of the needle tube 421, including the distal needle end 423, resides within the sealed chamber 438 which is otherwise filled with air. The needle tube 421 is provided with a distal side hole 425 and a proximal side hole 427, and the lumen of the needle tube 421 is therefore divided into a proximal flow channel 424, leading from the proximal needle end 422 to the proximal side hole 427, and a distal flow channel 426, leading from the distal side hole 425 to the distal needle end 423. However, in contrast to the fourth embodiment of the invention, the lumen of the needle tube 421 between the proximal side hole 427 and the distal side hole 425 comprises a block 429 which prevents fluid flow through the needle tube 421 between the proximal side hole 427 and the distal side hole 425.

The axial distance between the distal side hole 425 and the proximal side hole 427 is correlated with the axial dimension of the sealed chamber 438 such that in at least one particular position of the needle tube 421 relative to the chamber structure 430 the distal side hole 425 is positioned in a distal end portion and the proximal side hole 427 is positioned in a proximal end portion of the sealed chamber 438.

The needle shield 450 and the base member 411 are capable of relative axial motion between an extended relative position in which both the distal needle end 423 and the distal side hole 425 are accommodated within the sealed chamber 438 and a retracted relative position (e.g. as shown in FIG. 12a) in which the distal needle end 423 protrudes through the chamber septum 439 and the distal side hole 425 as well as the proximal side hole 427 are accommodated within the sealed chamber 438. The needle shield 450 and the base member 411 are biased towards the extended relative position by a compression spring 419.

The relative motion between the needle shield 450 and the base member 411 is enabled by means identical to the ones described in connection with the first embodiment of the invention. Consequently, a detailed description of this relative motion in connection with the present embodiment will be omitted.

FIG. 12a shows the needle unit 410 in an initial state during a very first drug injection event. The user has placed the needle unit 410 against the skin and pressed the injection device towards the skin to thereby bring the needle shield 450 and the base member 411 to the shown retracted relative position in which the distal needle end 423 protrudes through the chamber septum 439 and resides in a subcutaneous compartment within the user (not shown). As the cartridge 460 becomes pressurised and liquid drug 466 resultantly flows through the proximal flow channel 424 the proximal side hole 427 will allow entry of the liquid drug 466 into a proximal end portion of the sealed chamber 438. In fact, the presence of the block 429 will force the liquid drug 466 from the proximal flow channel 424 into the sealed chamber 438, and since the liquid drug 466 thus initiates a filling of the sealed chamber 438 from the proximal end portion thereof the air originally present in the sealed chamber 438 is gradually forced out through the distal side hole 425 (FIG. 12b), whereby the sealed chamber 438 becomes practically completely de-aerated as the liquid drug 466 continues out through the distal side hole 425, into the distal flow channel 426 and through the distal needle end 423 (FIG. 12c).

Hence, following the very first injection action and retraction of the injection needle 420 from the skin the distal portion of the needle tube 321 is practically submerged in preservative containing liquid drug 466 within the sealed chamber 438, which preservative containing liquid drug 466 provides a biostatic environment for this portion of the needle tube 421 in between-use periods. In this case any subsequent injection action will flush the sealed chamber 438 and in accordance with the first-in-first-out principle completely, or substantially completely, renew the liquid drug 466 therein to maintain an adequate level of microbacterial growth inhibiting substance over time.

In the between-use state of the needle unit 410 the distal side hole 425 is positioned within the sealed chamber 438, whereas the proximal side hole 427 and the majority of the portion of the needle tube 421 that defines the intermediate flow channel 428 are positioned in the sealing sleeve 440. However, the immobilised Zinc (Zi⁺⁺) in the sealing sleeve 440 will provide for neutralisation of potential micro-bacterial contaminants in that area even though the preservative containing drug 466 is not in permanent contact therewith.

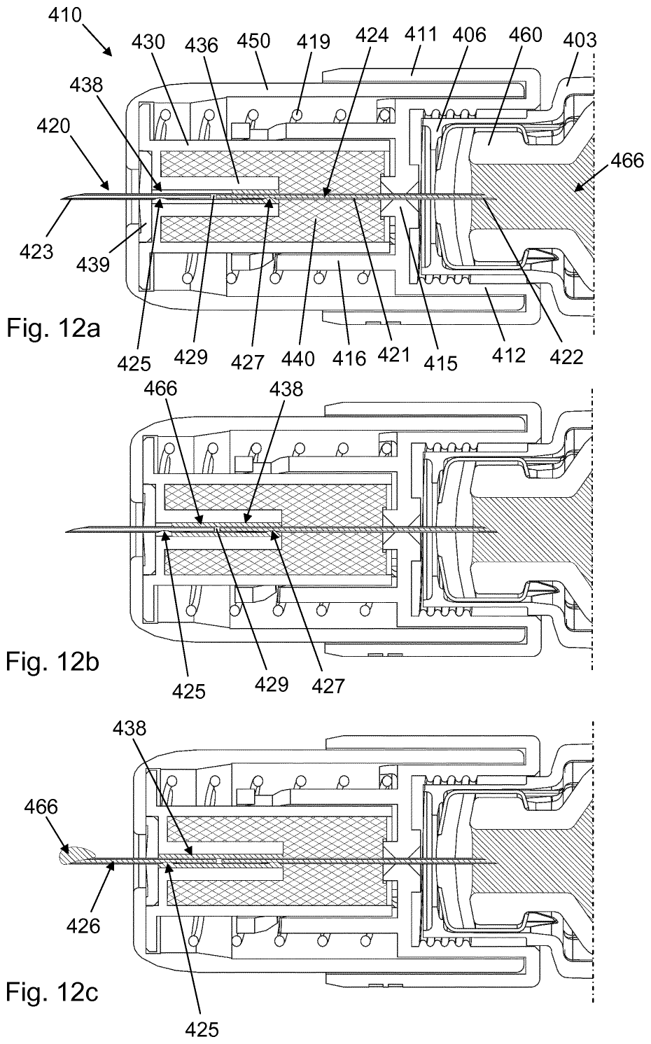

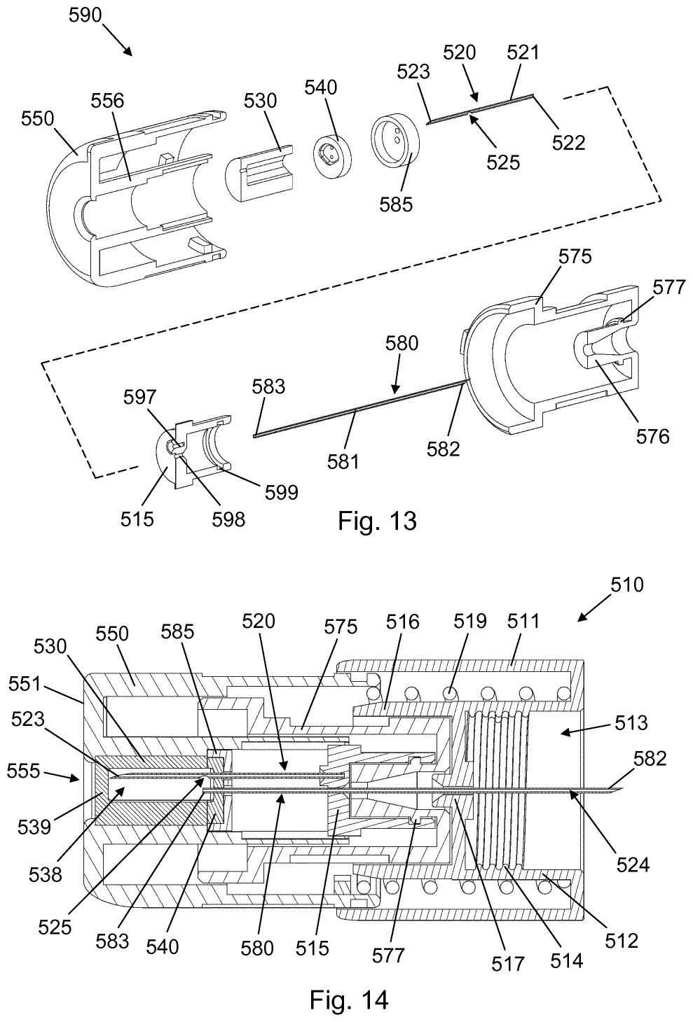

FIG. 13 is an exploded, partly longitudinally sectioned, view of a chamber sub-assembly 590 as used in a needle unit 510 (ref. FIG. 14) according to a sixth embodiment of the invention. The chamber sub-assembly 590 comprises a needle hub 515 and a hub support 575 comprising a hub carrier 576 with a circumferential protrusion 577 configured to engage with a circumferential groove 599 in the needle hub 515 to thereby axially fixate the needle hub 515 in the hub support 575.

The needle hub 515 has a through-going bore 598, in which an inlet needle 580 is fixedly mounted, and a seat 597 for reception and retention of an injection needle 520. The inlet needle 580 comprises an inlet needle tube 581 which has a pointed proximal inlet needle end 582 configured for penetration of a drug reservoir septum and a distal inlet needle end 583. The inlet needle tube 581 extends axially through the hub carrier 576, and the proximal inlet needle end 582 is thus positioned proximally of the hub support 575 while the distal inlet needle end 583 is positioned distally of the through-going bore 598.

The injection needle 520 comprises a needle tube 521 with a proximal needle end 522 which sits in the seat 597, a distal needle end 523 configured for penetration of a skin barrier, and a side hole 525. The chamber sub-assembly 590 further comprises a needle support 585, a chamber structure 530, a sealing disc 540, and a needle shield 550 having an axially extending chamber support member 556.

FIG. 14 is a longitudinal section view of the needle unit 510 in a pre-use state. The needle unit 510 consists of a base member 511, the chamber sub-assembly 590, and a compression spring 519. The base member 511 comprises an inner section 512, which defines a proximal space 513 and carries a thread 514 for reception of a needle mount 6 (ref. FIG. 5) of the injection pen 1, and a distal cup shaped structure 516 configured for reception of the hub support 575. The inner section 512 further comprises a transversal wall 517 separating the proximal space 513 and the cup shaped structure 516. The transversal wall 517 has a central bore through which the inlet needle 580 extends.

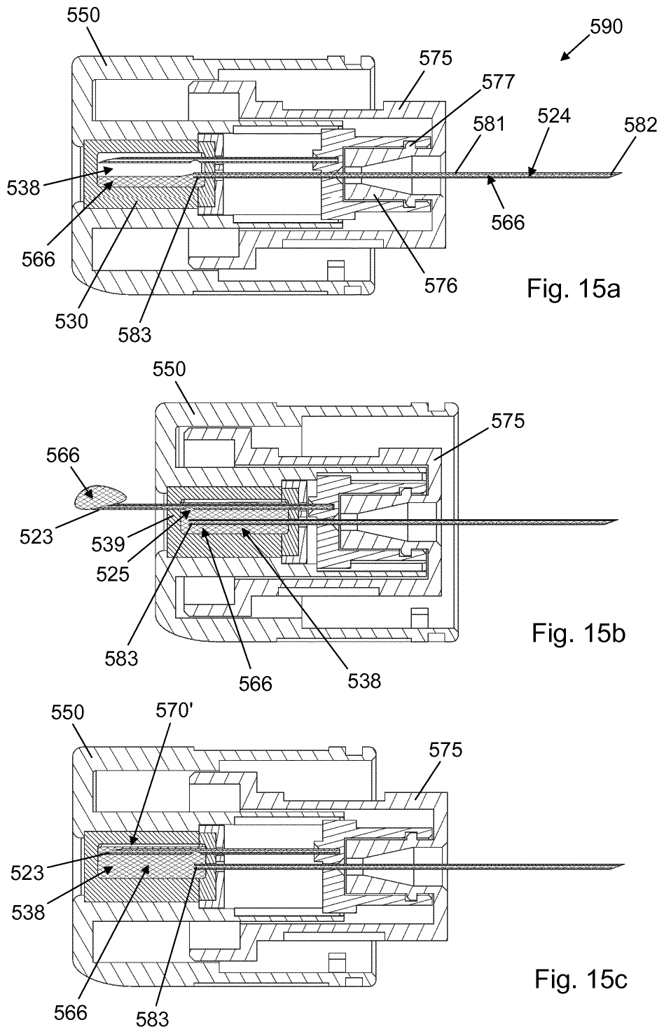

The chamber structure 530 has a penetrable self-sealing chamber septum 539, and defines, together with the sealing sleeve 540, a sealed chamber 538, which is fixedly arranged in a distal end portion of the needle shield 550. The inlet needle 580 extends from the proximal pace 513 through the through-going bore 598 in the needle hub 515 and respective dedicated bores in the needle support 585 and the sealing disc 540, and has the distal inlet needle end 583 positioned in a proximal end portion of the sealed chamber 538. Thereby, an inlet channel 524 is provided between the proximal space 513 and the sealed chamber 538, enabling flow of a preservative containing liquid drug 566 (ref. FIG. 15a) from a received injection pen 1 to the sealed chamber 538.

The injection needle 520 extends from the seat 597 through respective dedicated bores in the needle support 585 and the sealing disc 540 and into the sealed chamber 538. In the shown pre-use state of the needle unit 510 the distal needle end 523 is positioned in a distal end portion and the side hole 525 is positioned in a proximal end portion of the sealed chamber 538.

The sealing sleeve 540 is made of a thermoplastic elastomer and contains immobilised Silver (Ag⁺) to neutralise micro-bacterial contaminants in needle surface areas that are not in permanent contact with the preservative containing liquid drug 566.

The needle shield 550 comprises a transversal contact face 551 configured for abutment with a skin section (not shown) of a user. The transversal contact face 551 has an orifice 555 therein through which a distal portion of the needle tube 521 extends during a dose injection action. The needle shield 550 and the base member 511 are capable of relative axial motion between an extended relative position (FIG. 14) in which both the distal needle end 523 and the side hole 525 are accommodated within the sealed chamber 538 and a retracted relative position in which the distal needle end 523 protrudes through the chamber septum 539 and the orifice 555 and the side hole 525 is accommodated within the sealed chamber 538. The needle shield 550 and the base member 511 are biased towards the extended relative position by the compression spring 519.

FIGS. 15a-15c show the principle of the establishment of a biostatic environment for the distal portion of the needle tube 521 which goes into the user during dose expelling events. For the sake of clarity only the chamber sub-assembly 590 is depicted in these figures.

In connection with a first use of the injection pen 1 the user initially prepares for a small dose to be expelled in order to prime the needle unit 510. This is done by operation of the dose dial button 4. A subsequent depression of the injection button 5 leads to a pressurisation of the liquid drug 566 in the injection pen 1 which then begins to flow through the proximal inlet needle end 582 and into the inlet channel 524. When the liquid drug 566 reaches the distal inlet needle end 583 it pours into the sealed chamber 538 and gradually fills the sealed chamber 538, while compressing the air originally present therein (FIG. 15a). When the priming dose has been transferred to the sealed chamber 538 the user, holding the injection pen 1 in one hand, presses the needle shield 550 proximally against the force of the compression spring 519 to allow the distal needle end 523 to penetrate the chamber septum 539 and become exposed to the surroundings. The compressed air in the sealed chamber 538 immediately expands through the side hole 525 and leaves the injection needle 520 via the distal needle end 523. The user then releases the needle shield 550 which is automatically returned to its initial position (the extended relative position) by the compression spring 519. The sealed chamber 538 is now practically filled with liquid drug 566 and the injection procedure can commence.

After having set the desired dose by operation of the dose dial button 4 the user places the contact face 551 on the skin surface at the chosen injection site and presses the housing 2 against the skin. This brings the needle shield 550 and the base member 511 from the extended relative position (indicated in FIG. 15a) to the retracted relative position (indicated in FIG. 15b), against the force of the compression spring 519 (not shown). At this point both the side hole 525 and the distal inlet needle end 583 are positioned in a distal end portion of the sealed chamber 538.

Activation of the dose expelling mechanism of the injection pen 1 by depression of the injection button 5 now causes the set dose to flow through the inlet channel 524 and into the sealed chamber where it forces the liquid drug 566 already present out through the side hole 525 into the needle tube 521 and out through distal needle end 523 (FIG. 15b). Hence, the sealed chamber 538 is flushed by the dose expelled from the injection pen 1, except from a small volume thereof which remains in the sealed chamber 538 at the end of the injection action.

When the injection procedure is over and the user removes the injection needle 520 from the skin by pulling back the injection pen 1 the compression spring 519 automatically brings the needle shield 550 and the base member 511 into the extended relative position where the distal needle end 523 is accommodated in the sealed chamber 538. FIG. 15c illustrates the resulting between-use storage condition of the distal portion of the needle tube 521 and the distal inlet needle end 583. It is seen that the interior of the needle tube 521 is filled with liquid drug 566 and that the exterior of the distal portion of the needle tube 521, which gets in contact with the users skin, is stored in a combination of the liquid drug 566 remaining in the sealed chamber 38 and drug fumes 570′. The preservatives in the liquid drug 566 and in the drug fumes 570′ inhibit micro-bacterial growth and a biostatic environment is thus established for the lumen as well as for the exterior surface of the distal portion of the needle tube 521.

During each subsequent injection action the sealed chamber 538 will be flushed and at the end a fresh volume of liquid drug 566 will remain therein. This reduces the risk of potential degradation of the biostatic environment over time due to diffusion of preservatives from the sealed chamber 538.

1. A needle unit having a proximal space adapted to accommodate a portion of a variable volume reservoir, the needle unit comprising:
a needle carrier,
a needle tube being fixed to the needle carrier and comprising a distal needle end for providing fluid communication to an injection site,
a needle shield carrying a sealed chamber for accommodating a distal portion of the needle tube, the sealed chamber being sealed distally by a penetrable self-sealing septum, and
a flow channel for establishing fluid communication between the sealed chamber and the proximal space,
wherein the needle shield and the needle carrier are capable of relative motion between a first relative position in which the sealed chamber houses the distal needle end, and a second relative position in which the distal needle end protrudes from the sealed chamber through the penetrable self-sealing septum, and
wherein the needle tube further comprises a side hole, the side hole being in fluid communication with the flow channel and positioned within the sealed chamber when the needle shield and the needle carrier are in the second relative position.

2. The needle unit according to claim 1, wherein the sealed chamber has a volume in the range.

3. The needle unit according to claim 1, wherein the sealed chamber has a volume in the range.

4. The needle unit according to claim 1,
wherein the sealed chamber comprises a proximal cylindrical zone, a distal cylindrical zone, and an intermediate conical zone tapering towards the distal cylindrical zone, and
wherein the side hole is positioned in the distal cylindrical zone when the needle shield and the needle carrier are in the second relative position.

5. The needle unit according to claim 1,
wherein the side hole has a sloping edge portion connecting an interior surface of the needle tube with an exterior surface of the needle tube, the sloping edge portion sloping in the proximal direction from the interior surface of the needle tube to the exterior surface of the needle tube,
and wherein the side hole is positioned in a distal portion of the sealed chamber when the needle shield and the needle carrier are in the second relative position.

6. The needle unit according to claim 1,
wherein the needle tube further comprises a second side hole, and
wherein the side hole and the second side hole are arranged such that the side hole is positioned in a distal portion of the sealed chamber and the second side hole is positioned in a proximal portion of the sealed chamber, when the needle shield and the needle carrier are in the second relative position.

7. The needle unit according to claim 1, wherein the sealed chamber is cylindrical and has an inner diameter in the range.

8. The needle unit according to claim 1,
wherein the flow channel comprises a proximal needle end configured to penetrate a reservoir septum.

9. The needle unit according to claim 8, wherein the flow channel constitutes a proximal portion of the needle tube, the proximal portion being fluidly connected with the distal portion.

10. The needle unit according to claim 8, wherein the flow channel comprises a second needle tube arranged in parallel with the needle tube and extending between the proximal space and the sealed chamber.

11. The needle unit according to claim 1, wherein the side hole is positioned in a proximal end portion of the sealed chamber, when the needle shield and the needle carrier are in the first relative position and in a distal end portion of the sealed chamber, when the needle shield and the needle carrier are in the second relative position.

12. The needle unit according to claim 1, further comprising a needle support arranged between the sealed chamber and the needle carrier, the needle support being configured to slidably receive at least one needle tube.

13. An injection system comprising an injection device 1 and a needle unit according to claim 1, the injection device 1 holding, or being adapted to hold, a preservative containing liquid drug.

14. The injection system according to claim 13, wherein the needle unit further comprises first coupling structure arranged within the proximal space, and the injection device comprises second coupling structure for mating connection with the first coupling structure.

15. A method of enabling establishment of a biostatic environment for an injection needle having a distal needle end and a side hole, the method comprising:
(i) fixing the injection needle to a needle carrier and providing fluid communication between the side hole and a receiving space capable of receiving a drug reservoir holding a preservative containing liquid,
(ii) providing a sealed chamber for accommodating a distal portion of the injection needle, wherein the sealed chamber
is sterilised and sealed distally by a penetrable self-sealing septum, and
has a volume in the range, and
(iii) arranging the sealed chamber and the needle carrier such that
they are capable of undergoing relative motion between a first relative position in which the sealed chamber houses the distal needle end, and a second relative position in which the distal needle end protrudes from the sealed chamber through the penetrable self-sealing septum, and
the side hole is positioned within the sealed chamber when the sealed chamber and the needle carrier are in the second relative position.
